(19) United States
(12) Patent Application Publication
(10) Pub. No.: US 2023/0201517 A1
(43) Pub. Date: Jun. 29, 2023

(54) PROGRAMMABLE INTERACTIVE SYSTEMS, METHODS AND MACHINE READABLE PROGRAMS TO AFFECT BEHAVIORAL PATTERNS

(71) Applicant: RIZKALLA, Michael, Adel, New York City, NY (US)

(72) Inventor: Michael Adel Rizkalla, New York City, NY (US)

(21) Appl. No.: 18/008,400

(22) PCT Filed: Jun. 3, 2021
(86) PCT No.: PCT/US2021/035808

§ 371 Date: Dec. 5, 2022

Related U.S. Application Data
(60) Provisional application No. 63/033,852, filed on Jun. 3, 2020.

(51) Int. Cl. A61M 21/00; H04R 1/08; H04R 1/34; H04R 1/02; G06F 3/01; G10L 15/18; G10L 15/22; G10L 25/63; G06F 3/16; A63H 3/00; A63H 3/28; G06K 7/14

(52) U.S. Cl. CPC A61M 21/00; H04R 1/08; H04R 1/342; H04R 1/028; G06F 3/016; G10L 15/18; G10L 15/22; G10L 25/63; G06F 3/165; A63H 3/003; A63H 3/28; G06K 7/1417; G06K 7/1413; G06V 40/168

(57) ABSTRACT

Implementations of a programmable interactive system to interact with a user to alter a user's behavioral patterns are provided. Such systems can include one or more of a processor, at least one input sensor operably coupled to the processor to sense at least one sensor input, at least one output device to output at least one stimulus to be observed by the user, a core unit to interact with the user, the core unit being operably coupled to the processor, and a non-transitory machine-readable medium embodying a set of machine readable instructions that, when executed by the processor, cause the system to detect one or more sensor inputs by way of the at least one sensor, analyze said at least one or more sensor inputs to identify a parameter describing the status of the user, and responsive to determining the parameter describing the status of the user, causing the at least one output device to change output of at least one stimulus that is observable by the user.

### CROSS-REFERENCE TO RELATED APPLICATIONS

This patent application claims the benefit of priority to U.S. Provisional Patent Application No. 63/033,852, filed Jun. 3, 2020. This patent application is also related to U.S. Design patent application No. 29/750,311, filed Sep. 12, 2020. Each of the foregoing patent applications is incorporated by reference herein for all purposes.

### FIELD OF TECHNOLOGY

This disclosure relates generally to behavior (e.g., sleep) monitoring systems, and, in some implementations, to a methods and/or systems of interactive and interchangeable personalities of an intelligent behavioral monitoring and educational device for a user, such as a child, to develop a desired routine.

### DESCRIPTION OF RELATED ART

Remote monitors for monitoring children, for example, from a second location in a residence, are commonplace. Various versions of such devices exist. The present application provides improvements over such devices, as set forth herein.

### SUMMARY OF THE DISCLOSURE

Example embodiments of the present disclosure set forth advantages over the prior art. Other features and/or advantages may become apparent from the description that follows.

In accordance with some aspects of the present disclosure, a programmable interactive system to interact with a user to alter a user's behavioral patterns is provided. Such a system can include one or more of a processor, at least one input sensor operably coupled to the processor to sense at least one sensor input, at least one output device to output at least one stimulus to be observed by the user, a core unit to interact with the user, the core unit being operably coupled to the processor, and a non-transitory machine-readable medium embodying a set of machine readable instructions that, when executed by the processor, cause the system to detect one or more sensor inputs by way of the at least one sensor, analyze said at least one or more sensor inputs to identify a parameter describing the status of the user, and responsive to determining the parameter describing the status of the user, causing the at least one output device to change output of at least one stimulus that is observable by the user. The disclosure also provides a core unit as described herein independently of the rest of the system, for example, including some or all of the circuitry required to operate the system.

In some implementations, the system can further include a docking unit configured to receive the core unit, wherein the docking unit and core unit are configured to communicate electronically with each other. If desired, the docking unit can include circuitry to project at least one visual output (such as lighting and/or a projected image) and/or emit sound when the docking unit is coupled to the core unit onto a target surface.

In some implementations, the non-transitory machine readable instructions further comprise instructions to output an audio output in synchronization with a visual output. For example, the system can be configured to synchronize the telling of a story by one component of the system with a light output, projected image(s) and/or background sounds through the same or a different component of the system.

In some implementations, one or more components of the system (e.g., docking unit, core unit) can define a parabolic surface and include a microphone disposed in a location of the parabolic surface to focus incoming sound waves toward the microphone to enhance the system's ability to detect sounds made by the user.

In some implementations, one or more of the core unit and the docking unit can include a reconfigurable exterior surface. For example, the reconfigurable exterior surface can include an outer layer formed in the shape of a three dimensional object that can be removed from a frame of the core unit. By way of further example, the system can include attachments that couple to the core unit and/or docking unit that are rigid or semi-rigid. The outer layer (or other attachable component) can include an identification tag that is detected by the core unit, wherein, responsive to detecting the identification tag, the processor selects machine readable code to execute that is unique to the selected outer layer or other attachable component, and can outputs at least one stimulus associated with the identification tag.

If desired, the identification tag can include an electronic identification tag including information stored thereon. For example, the electronic identification tag can include one or more of a NFC chip or a RFID chip including digital information stored thereon. By way of further example, the identification tag can additionally or alternatively include an optical identification tag including information encoded therein. For example, the can include a QR code, or a bar code. By way of further example, the identification tag can additionally or alternatively include at least one visual indicium, such as a hologram, colored shape, a raised or lowered surface feature, such as bumps, divots, ridges or grooves, or can comprise a deflectable switch in a unique location, as desired.

In another implementation, the outer layer can include an identification tag that is detectable by a portable electronic device, wherein, responsive to detecting the identification tag, the processor selects and processes a discrete set of machine readable instructions unique to the identification tag. If desired, the processor can then output at least one visual or auditory stimulus associated with the identification tag. For example, the portable electronic device can be a smart phone. Responsive to detecting the identification tag, the smart phone can access and download electronic files through a network connection and copy them to or install them on the core unit, or another component of the system.

In some implementations, the system can include a plurality of different removable outer layers, wherein each said different removable outer layer is configured to be received by the core unit. Each said removable outer layer can have a unique identification tag, wherein each said unique identification tag is identified by the system when the removable outer layer including said unique identification tag is mounted on the core unit. Upon identifying said unique identification tag, a predetermined set of machine readable instructions specific to said unique identification tag can be selected by the processor to determine a visual and/or auditory output by the system.

In some embodiments, each of the plurality of different removable outer layers can have the appearance of a unique three dimensional figurine. Responsive to identifying said unique identification tag, the system can select machine readable code that includes information to cause the core unit to adopt behavioral characteristics associated with the unique three dimensional figurine.

In some implementations, the unique three dimensional figurine resulting from the removable outer layer can corresponds to a unique action figure. For example, the figurine can correspond to a cartoon character, a toy in a toy line, and the like. A plurality of unique outer layers can be provided with unique machine readable indicia so that, if a particular outer layer is applied to the core unit, the system is configured to access machine readable code that causes the system to express the traits of a character associated with the outer layer. Thus, if the removable outer layer corresponds to a well known cartoon character or actual person, the core unit can access machine readable code to permit it to speak in a voice that resembles that of the character and utter catch phrases of the character. Routines can then be executed that causes interaction between the user and the system, such as the system can read a bedtime story to the user in the voice of the character, and the like. As such, the system can accordingly provide additional functionality responsive to detecting mounting of a selected unique removable outer layer to the core unit.

In some implementations, the system can be configured to access updated configuration information from a remote server. The updated configuration information can include new visual and/or audio information to project to the user. Visual information can include light patterns, video, animations, and the like.

In some implementations, the core unit can be coupled to at least one processor, at least one memory, and at least one database. One or more of the at least one processor, at least one memory, and at least one database can be onboard the core unit. The core unit can include one or more of at least one camera, at least one battery, at least one sensor, and at least one infrared detecting sensor. The core unit can include a visual projector therein and a projection screen forming a surface thereof, wherein the visual projector projects an image onto the projection screen responsive to user input. The projection screen can be at least partially planar in shape as a flat and or curved plane. Alternatively, the projection screen may not be planar in shape. If desired, the projection screen can be at least partially spherical or spheroidal in shape. The projection screen can include at least one section of compound curvature. The projection screen can be at least partially formed by an intersection of curved surfaces.

In accordance with further aspects of the disclosure, the core unit can include a haptic controller to process haptic input detected by sensors of the core unit. If desired, the machine readable instructions can include instructions to recognize facial features or voice characteristics of the user. Upon recognizing a user, the system can load a profile file including settings and/or preferences of the user. The machine readable instructions can include instructions to interact with and respond to the user using natural language processing in real-time. The machine readable instructions can include instructions to generate an audiovisual response in response to the status of the user. If desired, the machine readable instructions can include a machine learning algorithm, for example, to improve interactive functions with the user. In some implementations, the system can be programmed to detect and analyze the user's voice to estimate the user's emotional state, and interact with the user by projecting a visual image responsive to the user's determined emotional state. The system can be programmed to detect and analyze the user's voice to estimate the user's emotional state, and respond to the user by projecting an audio segment responsive to the user's determined emotional state.

In some implementations, the system can further include a sleep management server that manages network resources by gathering data inputs related to sleep behavior of the user, analyzes the data inputs, and generates and sends at least one output to the user. The at least one output can include a recommendation to aid in sleep management decision-making for the user. The sleep management server can include machine readable instructions to maintain a real-time activity log to help develop and monitor a bedtime habit training of the user. The sleep management server can include instructions to provide a sleeping quality analysis of the user using a machine learning algorithm.

In some embodiments, the system can include a plurality of peripheral devices configured to communicate wirelessly with the processor. The system can be configured to detect using the at least one sensor when the user is restless or awakened. The at least one sensor can include at least one of a camera, a motion sensor, and a microphone. Responsive to determining if the user is restless or awakened, the system can be configured to play soothing audio output to help the user return to sleep.

In some implementations, the system can be configured to launch an interactive routine and interact with the user during the interactive routine. The routine can be a bedtime routine and the system can project lighting conducive to sleeping during the interactive bedtime routine. Likewise, the routine can be a bedtime routine and the system can project sounds conducive to sleeping during the interactive bedtime routine. If desired, the system can alter the routine in response to detecting the state of the user.

In some implementations, the system can engage in a gamified routine to achieve a goal by the user. The goal can be, for example, a task, and the system can provide instructions to the user to achieve the household task as the system detects the user taking actions in support of completing the task. For example, the task can include a household task such as setting a table, getting a drink of water, turning off lights, caring for a pet, reading a story and the like. The task can be to play a game, such as hide and seek, and the like.

In some implementations, the system can be configured to launch an interactive wakeup routine and interact with the user during the interactive wakeup routine. The system can project lighting conducive to waking up during the interactive wakeup routine. The system can project sounds conducive to waking up during the interactive wakeup routine.

In some implementations, the system can be configured to emit synchronized sounds or light from at least one further peripheral device and the core unit when the at least one further peripheral device is within a predetermined proximity of the core unit. The at least one further peripheral device and the core unit can provide complementary functions.

In some implementations, the system can engage in a gamified routine to facilitate interaction of a plurality of users. Each said user can be associated with a respective core unit, and each core unit can include a removable cover that resembles a unique three dimensional shape. To prevent communication interference, if desired, the core units may be assigned a hierarchy by the system, or one core unit can control the actions of a second or subsequent core unit. The gamified routine can include a role playing routine.

In some implementations, the machine readable instructions can further include instructions to determine a specific sleep state of the user. The machine readable instructions can further include instructions to read a narrative to the user while providing synchronized background sounds and lighting. If desired, the machine readable instructions can further include instructions to play predetermined sounds during a bedtime routine, and to play said predetermined sound again if the system determines that the user is awakening during a predetermined time period. The machine readable instructions can further include instructions to determine the developmental level of the user, and to provide audio and visual outputs responsive to the determined developmental level of the user. In some embodiments, the machine readable instructions can further include instructions to communicate with at least one peripheral device to obtain sensory inputs from the at least one peripheral device. The at least one peripheral can include a bath toy, and the system can obtains bath water temperature input, and/or other inputs, from the at least one peripheral device.

In some implementations, the machine readable instructions further include instructions to communicate with at least one peripheral device to obtain location information from said at least one peripheral device.

The system further includes a non-transitory machine-readable medium embodying a set of machine readable instructions that, when executed by a processor, cause the processor to carry forth any method described herein.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are illustrative and explanatory only and are not restrictive of the claims; rather the claims should be entitled to their full breadth of scope, including equivalents.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a schematic view of a sleep management server to manage sleep behavior of a child using an intelligent sleeping device communicatively coupled to the sleep management server through a computer network, according to one embodiment.

FIG. 2 is an exploded view of the intelligent sleeping device of the sleep management system of FIG. 1 illustrating a swappable robotic skin configured to enclose the automated core unit to acquire a robotic personality, according to one embodiment.

FIG. 3 is a block diagram of the intelligent sleeping device of the sleep management server of FIG. 1, according to one embodiment.

FIG. 4 is a conceptual view of the intelligent sleeping device of FIG. 1 illustrating the real-time animation projected by the integrated docking unit based on the swappable robotic skin of the intelligent sleeping device, according to one embodiment.

FIG. 5 is a conceptual view of the sleep management server of FIG. 1 illustrating the robotic personality of the intelligent sleeping device communicatively coupled to a mobile device responding to the child in real-time, according to one embodiment.

FIG. 6A is an implementation view of the sleep management system of FIG. 1 illustrating the intelligent sleeping device communicatively coupled to a mobile device encouraging the child to follow a nighttime routine in real-time, according to one embodiment.

FIG. 6B is a continuation of the implementation view of FIG. 6A illustrating the next steps of the child to follow the nighttime routine, according to one embodiment.

FIG. 6C is a continuation of the implementation view of FIG. 6B illustrating the next steps of the child to follow the nighttime routine, according to one embodiment.

FIG. 7 is another conceptual view of the sleep management system of FIG. 1 illustrating the night light phenomena created by the intelligent sleeping device, according to one embodiment.

FIG. 8 is a conceptual view of the sleep management system of FIG. 1 illustrating the rear projection mapping on a curved surface by the intelligent sleeping device, according to one embodiment.

FIGS. 9A-9B are an isometric cutaway view and a side cross sectional view of a core unit in accordance with the present disclosure indicating relative placement of an internal projector to a projection screen on a surface of the core unit.

FIGS. 10A-10C are views of a robotic skin and a core unit in accordance with the present disclosure

FIG. 11 and FIG. 12 provide diagrams of further representative embodiments of systems in accordance with the present disclosure.

Other features of the present embodiments will be apparent from the accompanying drawings and from the detailed description that follows.

### DETAILED DESCRIPTION

Example embodiments, as described below, may be used to provide a method and/or a system of creating an intelligent sleeping device to develop a nighttime routine for a child.

A sleeping device may be used to monitor a child's sleeping behavior. A child may not fully understand the concept of time. For example, the child may not understand when it is time for bed and when it is time to wake up. The sleeping device may be set to a desired sleep time to enable a child to sleep and wake at a set time. However, the sleeping device may produce harsh beeping sound and/or light, making the child irritated. Further, the child may be unable to interact with the sleeping device. The sleeping device may not be programmed to perform various activities according to the child's requirement and/or mood.

The sleeping device may be a programmable device of specific form designed to perform a particular function of monitoring the child's sleep behavior. However, the specific functionality of the programmable sleeping device may not be changed or improved to have a desirable qualities and/or function, resulting in a restricted usage of the programmable sleeping device

Disclosed are a method and/or a system of interactive and interchangeable personalities of an intelligent sleeping device for a child.

In one aspect, the disclosed intelligent sleeping device includes a method and system to create a robotic personality to aid in a bedtime habit training of a child. The robotic personality of the disclosed intelligent sleeping device may interactively initiate and progressively evolve a nighttime routine for the child to improve his or her sleep behavior. The robotic personality of the disclosed intelligent sleeping device may project a set of timed events to produce a calming environment for the child to wind down to prepare for a sound sleep. The robotic personality of the disclosed intelligent sleeping device may be a smart sleep companion for the child to help him get to sleep.

The robotic personality of the disclosed intelligent sleeping device may include circuitry associated with core functionalities relevant to a robot, and a number of swappable robotic skins. The robotic personality of the disclosed intelligent sleeping device may include an integrated docking unit, an automated core unit, and a swappable robotic skin. The disclosed intelligent sleeping device may be assembled by plugging-in the automated core unit to the integrated docking unit.

The robotic personality of the disclosed intelligent sleeping device may be configured to create a system to manage the bedtime routine for the child such that the child is encouraged to follow a wind down routine and go to bed at a preset time every day. The disclosed intelligent sleeping device may project a soothing light with music and/or animation to create a night environment to help the child doze off and gradually fall to sleep. The disclosed intelligent sleeping device may be configured to gamify the wind down activities and interact with the child to manage the nighttime routine of the child. In addition, the disclosed intelligent sleeping device may include a wake-up light alarm clock to simulate the sunrise to wake the child gently and naturally without harsh beeping sound.

The automated core unit of the disclosed intelligent sleeping device may include a robotic processor, a robotic memory, a robotic database, a camera, a speaker, a battery, and multiple sensors. The robotic processor may have audiovisual capabilities, including facial and voice recognition program. The robotic personality of the disclosed intelligent sleeping device may interact and respond to the child and/or a parent using natural language processing in real-time. The robotic personality may generate the audiovisual response based on the captured visual and auditory expression of the child and/or its parent.

The integrated docking unit may be a miniature dome-like structure with associated circuitry to project night light and/or animation when connected to the automated core unit. The integrated docking unit may be configured to project the colorful visuals of rainbows, clouds, smiling animated faces and angelic figures filling the room to create a nighttime experience for the child. In addition, the integrated docking unit may be configured to accompany the enthralling visuals with soothing and calming audio to create a tranquil surrounding to help lull the child to sleep.

The disclosed intelligent sleeping device may include a smart speaker with a set of timed events that are controlled and dispersed by a character on the smart speaker. When it is time for sleep, a beautiful light is projected by the integrated docking unit of the disclosed intelligent sleeping device and a select music (e.g., using Spotify, YouTube, apple music, amazon prime, mix, etc. connected to the smart device) may start to play to lull the child to sleep.

The automated core unit and the integrated docking unit may be connected over a wide area network (e,g, Internet) and/or a local area network (e.g., Wi-Fi). In addition, the automated core unit may include a proximity sensor to automatically detect and sync with the integrated docking unit to enable the robotic personality to activate.

The disclosed intelligent sleeping device may acquire a different personality based on various robotic skin characters. Each of the swappable robotic skin is configured with data related to a specific set of functionalities associated with a specific persona. The robotic personality may be automatically customizable for each of the specific personae associated with the configured number of swappable robotic skins.

The swappable robotic skins may be removably coupled to the automated core unit. Once coupled, the resulting robotic personality may be capable of performing the specific set of functionalities associated with each of the specific personae through a processor associated with the automated core unit and/or the configured corresponding swappable robotic skin.

The disclosed swappable robotic skin may be made of a stretchable silicon (e.g., silicone) sheet or molding that may give a frosting look to the intelligent sleeping device. In addition, the swappable robotic skins may include a haptic controller to respond to user's interactive activity (e.g., touch and motion etc.).

For example, the disclosed intelligent sleeping device may acquire a robotic personality of a panda when swappable robotic skin in the form of a teddy bear is removably coupled to the automated core unit. Once the swappable robotic skin in the form of the panda is plugged into the automated core unit, an RFID chip integrated in the robotic skin is activated and allows the robotic skin to sync with the automated core unit. Upon synching, the disclosed intelligent sleeping device may project an animated character of a panda and/or interact with the child. The animated character of the panda may playfully interact with the child to encourage him to follow a preset wind down routine in a fun way and thus, help him go to sleep.

The disclosed intelligent sleeping device may be configured to train the child into self-learning his sleep routine. The disclosed intelligent sleeping device may destress the parents and children as they engage in the sleep routine. The disclosed intelligent sleeping device may further monitor and help the child to stay asleep while sleeping during nighttime.

In another aspect, the automated core unit may be a programmable device to acquire a robotic personality when plugged into the integrated docking unit. The disclosed intelligent sleeping device may be communicatively coupled with a sleep management server through a wide area network. The disclosed intelligent sleeping device may be communicatively coupled to a plurality of mobile devices through a near field network. The sleep management server may keep a log of each of the child's sleep routines through the intelligent sleeping device.

The sleep management server may further map out the routine sleep activities of the child to improve its sleep behavior. The plurality of mobile devices coupled to the disclosed intelligent sleeping device may receive sleeping quality analysis of the child from the sleep management server. The sleeping quality analysis of the child may help improve the sleep behavior, sleep training, sleep correcting, sleep understanding, and wake up routine of the child. In addition, the sleep management server may provide a subscription based parenting support.

In yet another aspect, the disclosed intelligent sleeping device may operate on the edge computing. The disclosed intelligent sleeping device may operate in a distributed, open IT architecture featuring decentralized processing power, enabling mobile computing and Internet of Things (IoT) technologies and then syncing with the cloud system. The disclosed intelligent sleeping device may act as a server. The edge computing system may enable the data to be processed by the disclosed intelligent sleeping device itself and/or by a local computer and/or server, rather than being transmitted to a data center (e.g., sleep management server). Accordingly, the disclosed intelligent sleeping device may itself act as the command center to automatically assist the parent in bedtime habit training of the child.

In yet one more further aspect, the disclosed swappable robotic skin may be a robotic shell made of a soft silicone material and/or a cloth. The soft silicone shell and/or clothing may include an RFID tag to identify which clothing the robot is wearing. The disclosed swappable robotic skin made of soft silicone shell and/or clothing with the RFID tag may help the automated core unit to change it from an intelligent sleep device to a licensed property and/or a new property all together The automated core unit may go inside any type of character and/or clothing.

In an additional aspect, the integrated docking unit may be programmed to turn an object, often circular and/or irregularly shaped small indoor objects (e.g., a globe, a semi-sphere, etc.), into a display surface for video projection. The integrated docking unit may use projection mapping to display and/or project animation and/or a video film on any curved surface. The rear projection mapping may allow the integrated docking unit to project accurately on curved surfaces, such as a globular structure and/or a curved screen. By “any curved surface”, it is implied that the face can be shaped to any character.

The methods and systems disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows.

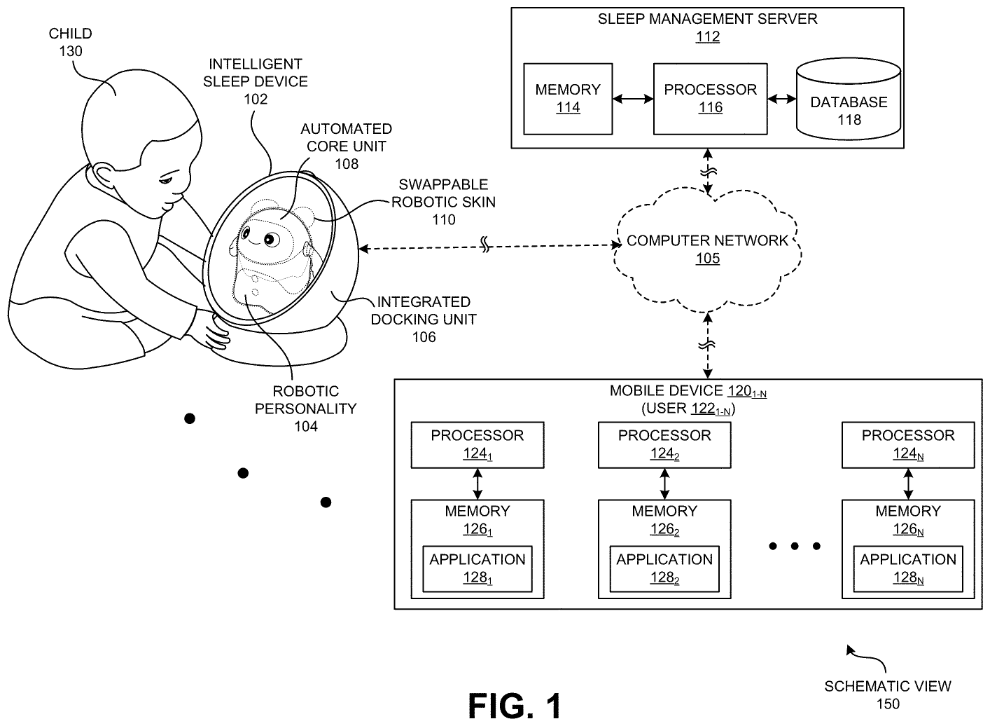

FIG. 1 is a schematic view 150 of a sleep management server 112 to manage sleep behavior of a child 130 using an intelligent sleeping device 102 communicatively coupled to the sleep management server 112 through a computer network 105, according to one embodiment. Particularly, FIG. 1 illustrates an intelligent sleeping device 102, a robotic personality 104, an integrated docking unit 106, an automated core unit 108, a swappable robotic skin 110, a sleep management server 112, a memory 114, a processor 116, a database 118, a computer network 105, a mobile device 120 (1-N), a child 130, a processor 124(1-N), a memory 126(1-N), and an application 128(1-N), according to one embodiment.

The intelligent sleeping device 102 may be an automated robotic machine designed to interactively monitor and improve a child's 130 sleep behavior by projecting a set of preprogrammed events to create a sleep environment. The intelligent sleeping device 102 may create a smart sleep companion (e.g., robotic personality 104) that may interact with the child 130 to develop a nighttime routine for the child 130.

The robotic personality 104 may be an automated character that interacts with the child 130 to encourage him to perform a set of activities and train him to follow a sleep routine. The robotic personality 104 may be programmed to capture the child's 130 voice and respond to the child by projecting an animated character based on the child's 130 mood according to the preprogrammed set of activities. The robotic personality 104 may use natural language processing (e.g., using machine learning algorithm 340) of the sleep management server 112 to respond to the child's voice in real-time.

The robotic personality 104 may physically and/or characteristically resemble a specific persona based on the character of swappable robotic skin 110. The robotic personality 104 may perform complex actions and/or operations associated with the particular persona. In one embodiment, robotic personality 104 may require the intelligent sleeping device 102 to virtually interact with a number of mobile devices 120(1-N) to realize multiple projection scenarios (e.g., an animation scenario, real-time projection 404) based on the user's recommendations 342.

The integrated docking unit 106 may be a base station of the robotic personality 104 designed to automatically display and project animation and/or soothing light to create a nighttime environment for the child 130. The integrated docking unit 106 may automatically sync with the automated core unit 108 through the local area network (e.g., a WIFI). Once synched, the integrated docking unit 106 may project and/or display animation (e.g., real-time projection 404) based on user's recommendations 342 and/or preprogrammed set of activities for the particular child 130. The user 122 may set a number of activities for the child 130 using a mobile device 120 communicatively connected to the sleep management server 112 through the computer network 105.

The automated core unit 108 may be an intelligent machine designed to capture the audiovisual interactive activities within its vicinity and respond based on the child's 130 mood and/or user's recommendations 342. The automated core unit 108 may capture the child's 130 voice through a microphone and/or visual activity through the camera 334 in real-time and virtually respond to the child 130 audio visually by projecting an animated character. The automated core unit 108 may include a smart speaker (e.g., mic with speaker) with a set of timed events that are controlled and dispersed by the robotic character on the smart speaker.

The swappable robotic skin 110 may be a virtual robotic character that may adapt to a particular character once connected to the automated core unit 108. The swappable robotic skin 110 may be the character that encloses the automated core unit 108. The swappable robotic skin 110 of the automated core unit 108 may be easily adaptable and could change personas (e.g., robotic personality 104) according to the physical character of the swappable robotic skin 110.

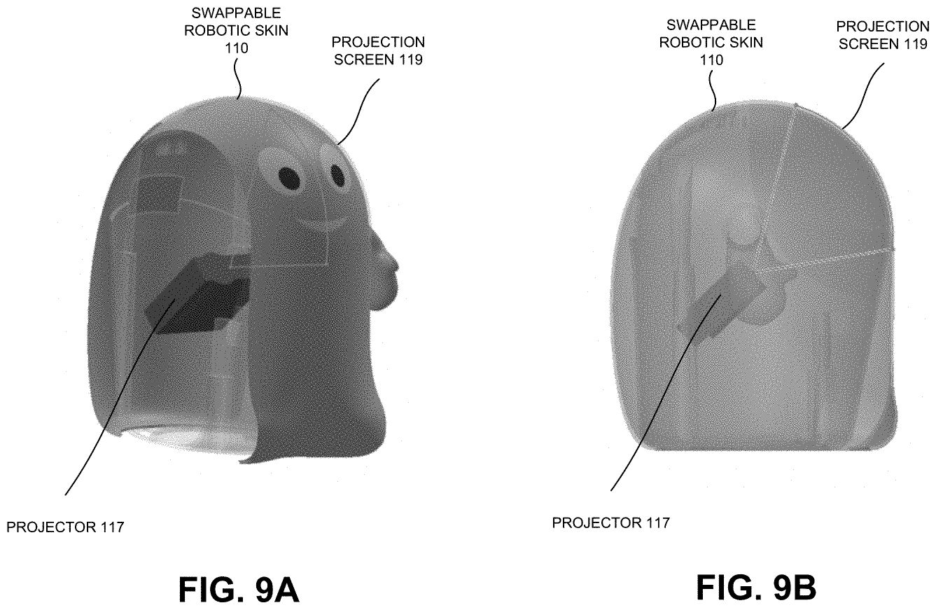

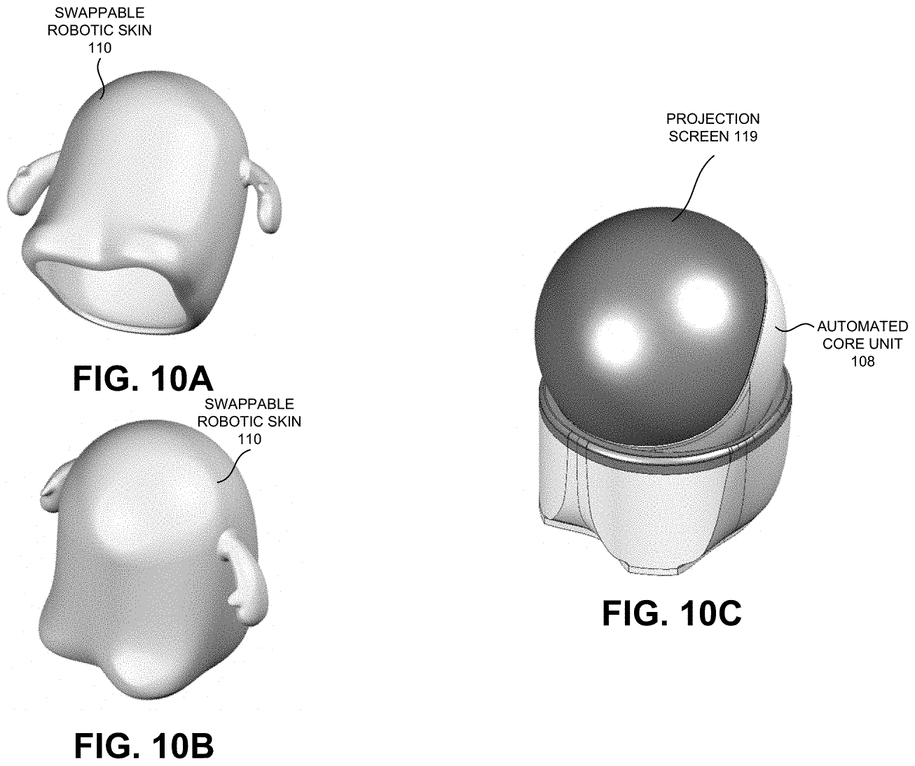

As illustrated in FIGS. 9A-9B, a projector 117 can be situated within the core unit 108 underneath the swappable skin 110. FIGS. 10A-10C illustrate a top front perspective view of the skin 110, a lower rear perspective view of the skin 110 showing a cavity inside the skin, and an isometric front view of the core unit 108, wherein the projection screen 119 is illustrated as being generally spherical in shape, but it will be appreciated that the screen can be any desired shape. The projector 117 inside the core unit 108 projects an image onto the screen 119, and this can cause the formation of facial features or other visual features on the skin 110, and can also provide moving indicia or features to simulate mouth movements associated with speaking, eye movement, emotional states, and the like.

In another embodiment, the disclosed swappable robotic skin 110 may be a robotic shell made of a soft silicone material and/or a cloth. The soft silicone shell (e.g., swappable robotic skin 110) and/or clothing (e.g., outfit 202) may include an RFID tag 338 to identify which clothing (e.g., outfit 202) the automated core unit 108 is wearing. The disclosed swappable robotic skin 110 made of soft silicone shell and/or clothing (e.g., outfit 202) with the RFID tag 338 may help the automated core unit 108 to change it from an intelligent sleep device 102 to a licensed property and/or a new property all together The automated core unit 108 may go inside any type of character and/or clothing (e.g., outfit 202).

The sleep management server 112 may be a computer program and/or a device in the computer network that manages network resources by gathering data related to sleep behavior from its multiple client devices (e.g., mobile device 120(1-N)) analyzes the information, and provides data, services, and/or programs to other client devices in the network. The sleep management server 112 may report data to aid in sleep management decision-making of a particular child 130.

In another embodiment, the disclosed intelligent sleeping device 102 may operate on the edge computing. The disclosed intelligent sleeping device 102 may operate in a distributed, open IT architecture featuring decentralized processing power, enabling mobile computing (e.g., using mobile device 120(1-N)) and Internet of Things (IoT) technologies and then syncing with the cloud system. The disclosed intelligent sleeping device 102 may act as a server. The edge computing system may enable the data to be processed by the disclosed intelligent sleeping device 102 itself and/or by a local computer and/or server, rather than being transmitted to a data center (e.g., sleep management server 112). Accordingly, the disclosed intelligent sleeping device 102 may itself act as the command center to automatically assist the parent in bedtime habit training of the child 130.

The memory 114 may be a storage space in the sleep management server 112, where data to be processed and instructions required for processing are stored. The memory 114 of the sleep management server 112 may store the robotic characteristics of the multiple robotic personality 104 (e.g., of the swappable robotic skin 110). The processor 116 may be a logic circuitry that responds to and processes the basic instructions to drive the sleep management server 112. The database 118 may be easily accessible to a large amount of information stored in the sleep management server 112.

The computer network 105 may refer to a variety of long-range and/or short-range (e.g., including near-field communication based networks) computer networks such as a Wide Area Network (WAN), a Local Area Network (LAN), a mobile communication network, WiFi, and Bluetooth®. Contextual applicability may be implied by the use of the term “computer network” with respect to computer network 105.

The computer network 105 may refer to Bluetooth® or mobile Internet when one or more device(s) 120(1-N) interacts with intelligent sleeping device 102. In another example, a WAN and/or a LAN may be employed for communication between sleep management server 112 and intelligent sleeping device 102.

The mobile device 120 (1-N) may be plurality of computing devices communicatively coupled to the intelligent sleeping device 102 through a local area network and/or a near field network (e.g., WIFI) to virtually interact with the intelligent sleeping device 102. The mobile device 120 (1-N) may further be communicatively coupled to the sleep management server 112 through a computer network 105.

Each mobile device 120 (1-N) may enable the mobile device user 122 (e.g., a child 130, a parent, a caretaker, etc.) to control the functionalities of the intelligent sleeping device 102, based on the robotic personality 104 of the robotic character of the swappable robotic skin 110. The mobile device 120 (1-N) may be provided with the augmented reality, the mixed reality and/or the virtual reality interactive experience. The mobile device 120 (1-N) may be a mobile phone, a personal computer, a tab, a laptop and/or any other network-enabled computing device, according to one embodiment.

The user 122(1-N) may be a person using the mobile device 120(1-N) to manipulate the intelligent sleeping device 102 to manage its child's sleep behavior using the intelligent sleeping device 102. The processor 124(1-N) may be a logic circuitry that responds to and processes the basic instructions to drive the mobile device 120 (1-N). The memory 126(1-N) may be a storage space in the mobile device 120 (1-N), where data is to be processed and instructions required for processing are stored. The application 128(1-N) may be a software program that runs on the mobile device 120 (1-N) and is designed to enhance the user productivity by managing the child's 130 sleep behavior using the intelligent sleeping device 102.

In an example embodiment, the intelligent sleeping device 102 may detect (e.g., using the sensors 326, camera 334 etc. of the automated core unit 108) that the child is restless and has woken up between his sleep and is crying. The intelligent sleeping device 102 communicatively coupled to the mobile device 120 may send a notification 504 to the sleep management server 112. The processor 116 of the sleep management server 112 may initiate the application 128(1-N) in the mobile device 120. The application 128(1-N) may send a notification 504 to the intelligent sleeping device 102 to play a soothing and calming audio (e.g., music 606) based on the user's recommendations 342 in the database 118 (e.g., using the machine learning algorithm 340) that creates a tranquil surrounding and helps lull the child 130 back to sleep.

The intelligent sleeping device 102 may play back appropriate animation and some appropriate music based on the user's recommendations 342 in the database 118 using real projection mapping.

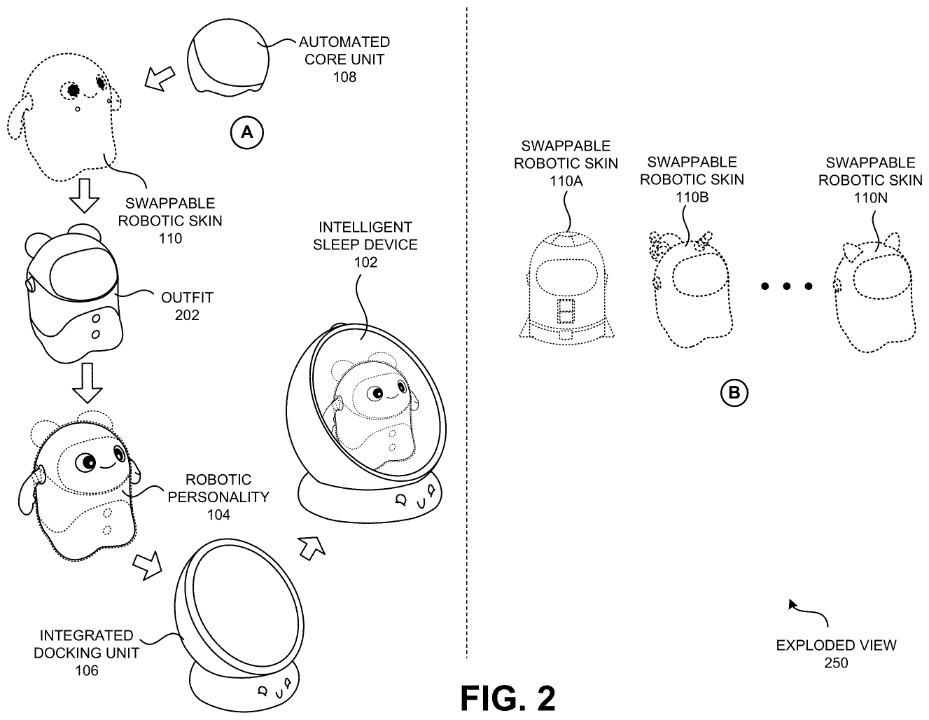

FIG. 2 is an exploded view 250 of the intelligent sleeping device 102 of the sleep management server 112 of FIG. 1 illustrating a swappable robotic skin 110 configured to enclose the automated core unit 108 to acquire a robotic personality 104, according to one embodiment. FIG. 2 shows a swappable robotic skin 110 made of a flexible silicone material configured to enclose the automated core unit 108. The swappable robotic skin 110 may be enveloped onto the automated core unit 108 as shown in circle ‘A’ of FIG. 2 and/or connected via a magnet to automated core unit 108 for a specific robotic personality 104, according to one embodiment.

In an alternate embodiment, the swappable robotic skin 110 may include a data port and upon plugging that data port into the automated core unit 108, the robotic personality 104 will inherit the personality of the robotic skin character. Circle ‘B’ of FIG. 2 illustrates a number of swappable robotic skin 110 depicting numerous robotic skin characters.

In one or more embodiments, the automated core unit 108 may be activated to perform operations associated with a specific robotic personality 104 relevant to a corresponding swappable robotic skin 110 based on plugging of the swappable robotic skin 110 onto automated core unit 108. In alternate implementations, swappable robotic skin 110 may be configured to receive automated core unit 108 therein.

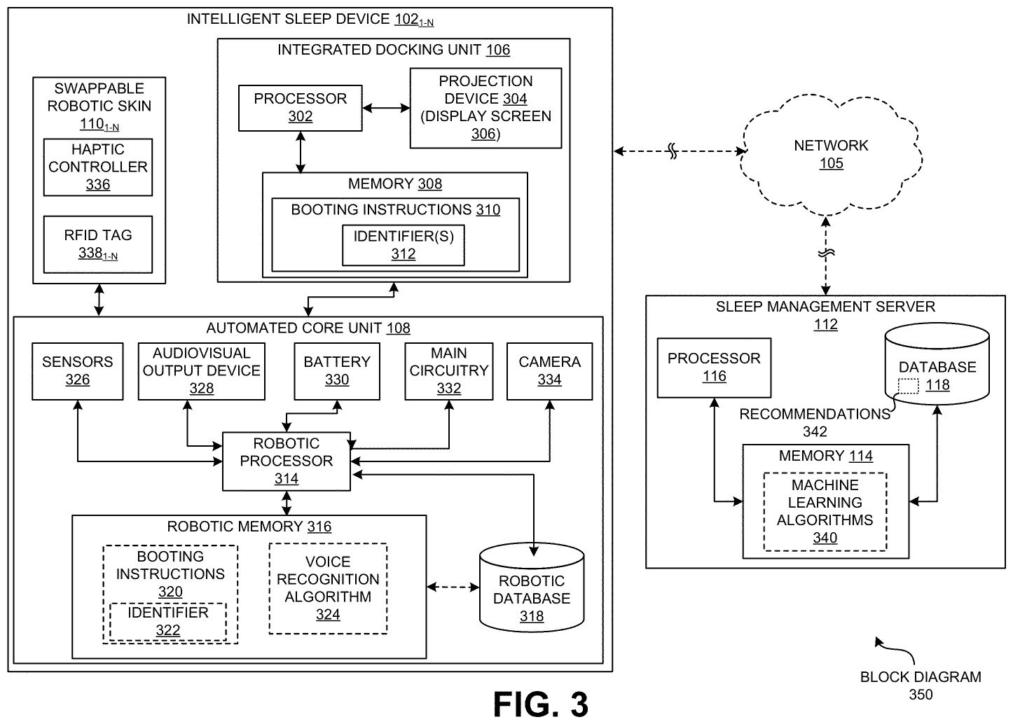

FIG. 3 is a block diagram 350 of the intelligent sleeping device 102 of the sleep management system of FIG. 1, according to one embodiment. Particularly, FIG. 1 builds on FIG. 2 and further adds, a processor 302, a projection device 304, a display screen 306, a memory 308, a booting instructions 310, an identifier 312, a robotic processor 314, a robotic memory 316, a robotic database 318, booting instructions 320, identifier 322, a voice recognition algorithm 324, a sensor 326, an audiovisual output device 328, a battery 330, a main circuitry 332, a camera 334, a haptic controller 336, an RFID tag 338 and a machine learning algorithm 340.

The processor 302 may be a logic circuitry that responds to and processes the basic instructions to drive the integrated docking unit 106. The projection device 304 of the integrated docking unit 106 may be an output device that can display motion pictures by projecting an image from them upon a screen of the integrated docking unit 106. The projection device 304 may take images generated by a computer and reproduce them by projecting onto the automated core unit 108 and/or another surface. The projection device 304 may project animation (e.g., real-time animation 404) and/or images of sky on the dome-like ceiling of the automated core unit 108 to create a nighttime experience for the child.

In another embodiment, the projection device 304 of the integrated docking unit 106 may be a handheld optical projector to provide virtual projection (VP). The projection device 304 of the integrated docking unit 106 may create an interaction metaphor by intuitively controlling the position, size, and orientation of a handheld optical projector's image.

The display screen 306 may be a surface area of the integrated docking unit 106 upon which text, graphics and video are temporarily made to appear for child's viewing. The internal surface 706 of the spherical dome 708 of the integrated docking unit 106 may act as a display screen 306 to display an animated graphic 704.

In an alternate embodiment, the external surface of the spherical dome 708 of the integrated docking unit 106 may act as a display screen 306 to display the northern light phenomena (e.g., northern light projection 702) to create an ethereal display of colored lights shimmering across the room for the child 130.

The memory 308 may be a storage space in the integrated docking unit 106, where data to be processed and instructions required for processing are stored. The booting instructions 310 may be an initial set of commands that the integrated docking unit 106 needs to perform when electrical power is switched on. The integrated docking unit 106 needs to perform the initial set of operations to sync with the automated core unit 108 to be ready to perform its normal operations.

For example, once the automated core unit 108 is within the communication range of the integrated docking unit 106, the booting instructions 310 may activate the integrated docking unit 106 to automatically sync with the automated core unit 108 to perform its various functionalities including projecting animation, colorful visuals of rainbows, stars, clouds, smiling faces and angelic figures, etc. to create a happy sleeping environment for the child.

The identifier 312 may be a unique attribute and/or name of a program or the names of the variables within a program that are used to identify the relevant data/info relevant to the integrated docking unit 106.

The robotic processor 314 may be a logic circuitry that responds to and processes the basic instructions to drive the automated core unit 108. The robotic memory 316 may be a storage space in the automated core unit 108, where data is to be processed and instructions required for processing are stored. The robotic database 318 may be a collection of information that is organized so that it can be easily accessed, managed and updated in the automated core unit 108.

The booting instructions 320 may be an initial set of commands that the automated core unit 108 needs to perform when electrical power is switched on. The automated core unit 108 needs to perform the initial set of operations to sync with the integrated docking unit 106 to be ready to perform its normal operations. The identifier 322 may be a unique attribute and/or name of a program or the names of the variables within a program that are used to identify the relevant data/info relevant to the automated core unit 108.

The voice recognition algorithm 324 may be a set of instructions that defines what needs to be done to identify a voice using a finite number of steps so as to respond to it auditorily, audio visually and/or animatedly in real-time based on the robotic personality 104 of the intelligent sleeping device 102. For example, the robotic personality 104 may simply speak to and/or respond to the child in real-time using the natural language processing and voice recognition algorithm 324 of the automated core unit 108.

The sensor 326 may be a device, module, machine, or subsystem whose purpose is to detect events and/or changes in its environment and send the information to the automated core unit 108. The automated core unit 108 may include a light sensor, a motion sensor, and/or a temperature sensor to automatically detect the changes in the surrounding environment to respond accordingly.

The audiovisual output device 328 may capture audio (sound) and/or visual (i.e. image or video) inputs, generating a signal that can be accessed by other devices. The battery 330 of the automated core unit 108 may supply the power to the automated core unit 108 when plugged in. The automated core unit 108 may receive power from the battery 330 to activate the automated core unit 108 of the intelligent sleeping device 102.

The automated core unit 108 may include the main circuitry 332 for functioning of the automated core unit 108. FIG. 3 shows the main circuitry 332 as interfaced with (and, thereby, controlled by) the robotic processor 314. In one or more embodiments, main circuitry 332 along with booting instructions 320 and a relevant wrapper may help assemble and activate the automated core unit 108 when a swappable robotic skin 110 is enveloped onto the automated core unit 108.

In one embodiment, the main circuitry 332 may be powered by the plugging in of the aforementioned swappable robotic skin 110 into automated core unit 108. For example, the plugging-in of the swappable robotic skin 110 into automated core unit 108 may provide electrical paths for a battery 330 (e.g., rechargeable) of automated core unit 108 to power main circuitry 332.

The camera 334 may be a vision system of the automated core unit 108 to find the child in its vicinity. Further, the camera 334 may enable the automated core unit 108 to determine the position and/or environmental condition in its vicinity. The camera 334 may capture and transmit the real-time visual signal to the wirelessly coupled number of mobile devices 120. In addition, the camera 334 may capture the real-time facial expression of the child operating the automated core unit 108 to enable the automated core unit 108 to generate the auditory response 506 based on the captured facial expression. The automated core unit 108 may generate the auditory response 506 and/or visual response 508 to project an animation based on the user's recommendations 342 in the database 118 using the machine learning algorithm 340, according to one embodiment.

The RFID tag 338 may be a set of digital data encoded in an integrated circuit and an antenna embedded in the swappable robotic skin 110. Each swappable robotic skin 110 may include an RFID tag 338 to identify the particular swappable robotic skin 110.

Once a particular swappable robotic skin 110 is placed onto the automated core unit 108, the radio frequency identification reader (RFID reader) may gather information from the RFID tag 338 using radio waves and capture the information stored on the tag. The RFID reader of the automated core unit 108 may send the unique identifier 322 of the particular swappable robotic skin 110 to the sleep management server 112. The sleep management server 112 may send a set of booting instructions 320 that correspond to the particular swappable robotic skin 110 to activate the robotic personality 104 analogous to the particular swappable robotic skin 110.

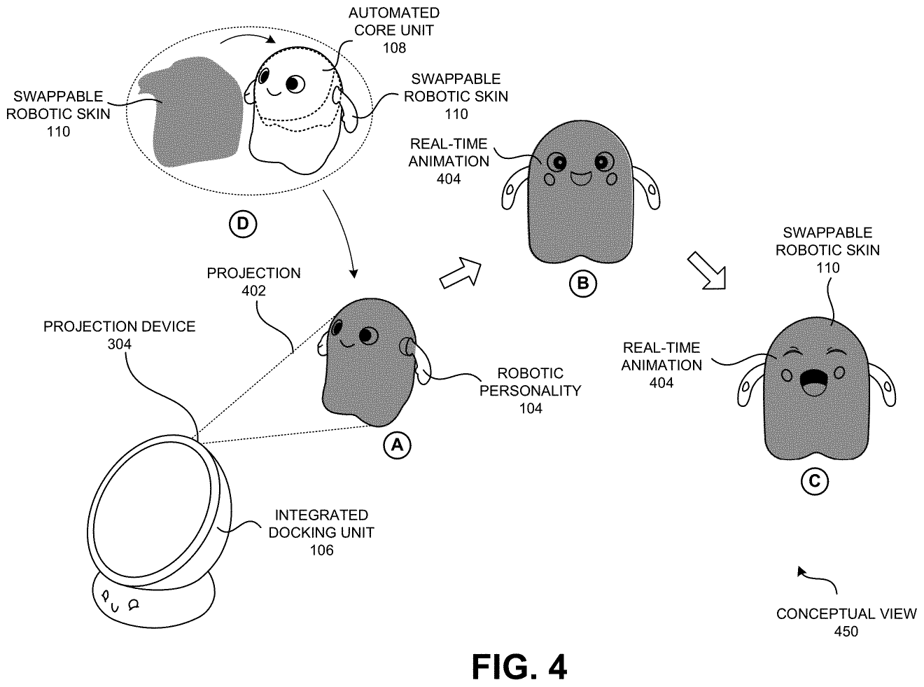

FIG. 4 is a conceptual view 450 of the intelligent sleeping device 102 of FIG. 1 illustrating the real-time animation 404 projected by the integrated docking unit 106 based on the swappable robotic skin 110 of the intelligent sleeping device 102, according to one embodiment.

As shown in FIG. 4, once the robotic personality 104 is within range of the integrated docking unit 106, the integrated docking unit 106 is automatically synched with the automated core unit 108. The projection device 304 of the integrated docking unit 106 projects little projection 402 on the automated core unit 108 to display an animated character (e.g., real-time animation 404) based on the swappable robotic skin 110 character enclosing the automated core unit 108 as shown in the circle ‘A’. The real-time animation 404 projected on the automated core unit 108 may respond and talk to the child as shown in the circle ‘B’ and ‘C’ of FIG. 4.

The projection device 304 of the integrated docking unit 106 may project from the top. The inside of the integrated docking unit 106 may include a decal that may light up by the projection coming from the projection device 304. The outside of the integrated docking unit 106 may have a light array that allows it to create a northern lights type effect (e.g., a moving light).

The disclosed swappable robotic skin 110 may be made of a stretchable silicon sheet (e.g., outfit 202) that may give a frosting look to the intelligent sleeping device 102 as shown in circle ‘D’ FIG. 4. In addition, the swappable robotic skins may include a haptic controller 336 to respond to child's interactive activity (e.g., touch and motion etc.).

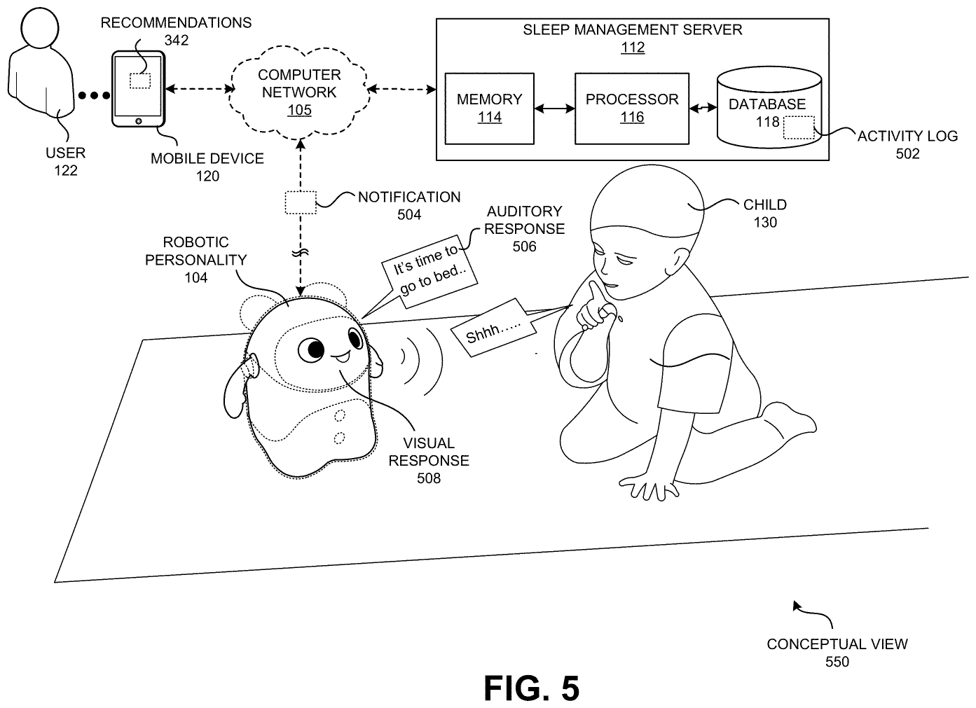

FIG. 5 is a conceptual view 550 of the sleep management system of FIG. 1 illustrating the robotic personality 104 of the intelligent sleeping device 102 communicatively coupled to a mobile device 120 interacting with the child 130 in real-time, according to one embodiment.

According to one embodiment, during the day the child 130 may have the robotic personality 104 in the house and be able to keep it with them as a toy (e.g., a teddy bear-type character). The robotic personality 104 may be separated from the integrated docking unit 106 to enable the robotic personality 104 to act as an interactive toy for the child 130 based on the particular swappable robotic skin 110 character.

As shown in FIG. 5, the camera 334 and sensors 326 of the automated core unit 108 may capture the child's voice and visual activity of the child 130 while the child 130 is playing with the robotic personality 104 during daytime. The robotic personality 104 may send a notification to the sleep management server 112. The child's activity log 502 is saved in the database 118 of the sleep management server 112. The auditory response 506 and visual response 508 is generated by the sleep management server 112 based on user's recommendations 122 in response to the child's activity. The robotic personality 104 may interactively relay the auditory response 506 and visual response 508 to the child in real-time.

The real-time activity log 502 of the sleep management server 112 may help a user 122 to develop and monitor a bedtime habit training of his child 130. The sleep management server 112 may provide a sleeping quality analysis of the child 130 using machine learning algorithm 340 to provide parenting support to the user 122. The child 130 may have super fun interaction with the robotic personality while developing a nighttime routine.

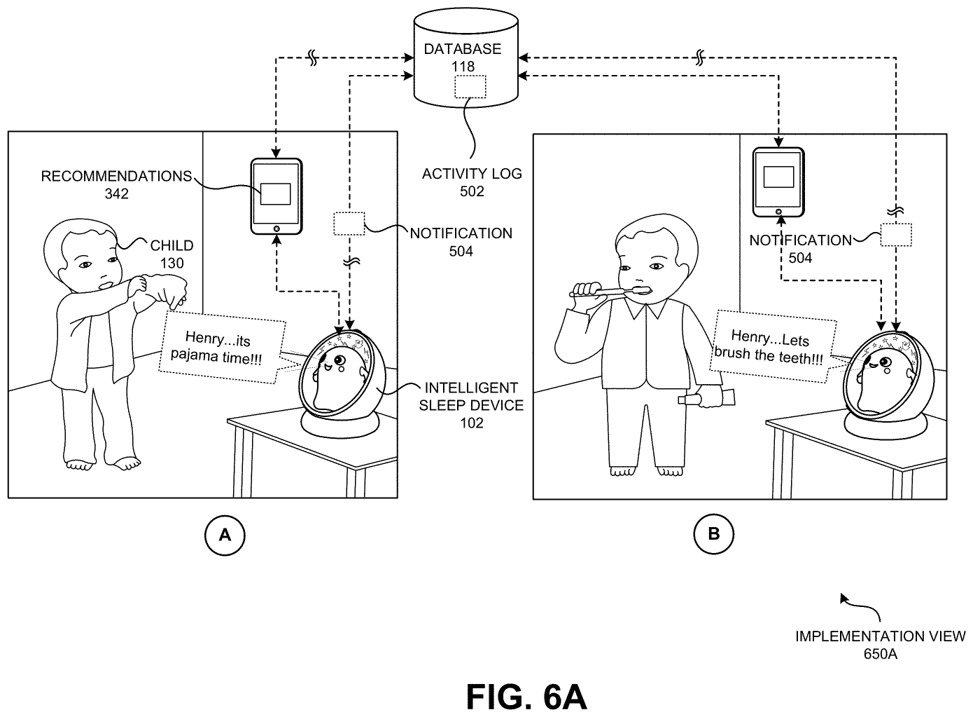

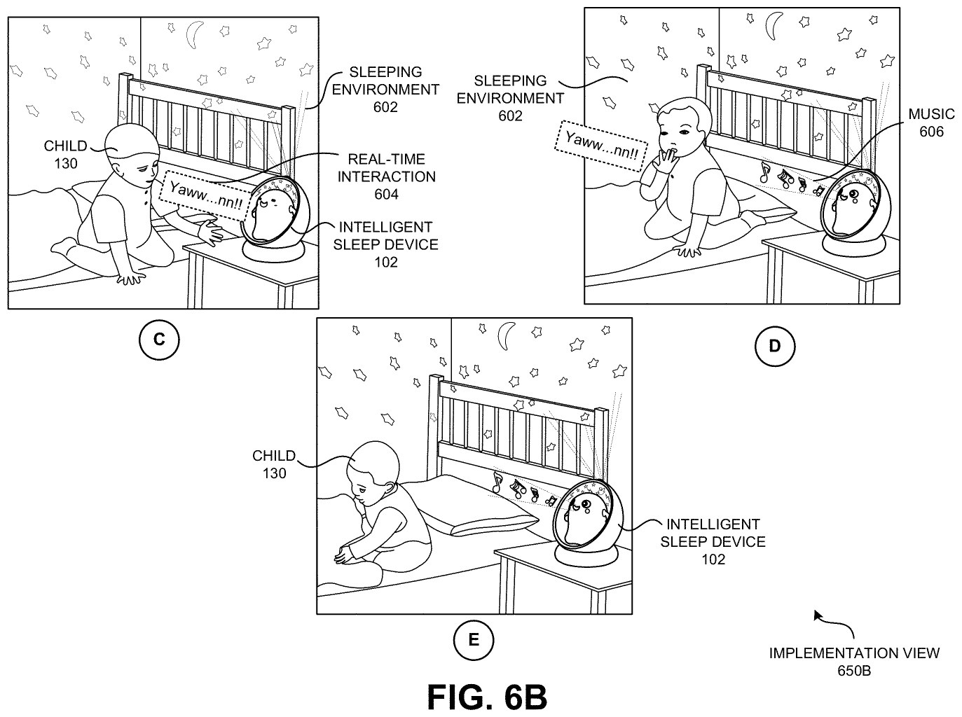

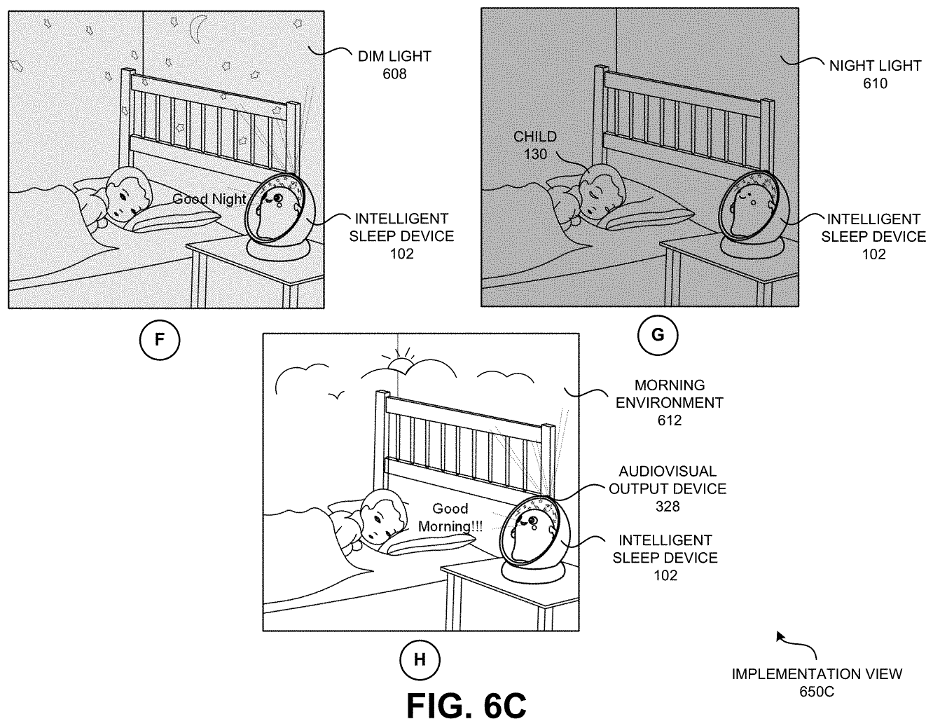

FIG. 6A is an implementation view 650A of the sleep management system of FIG. 1 illustrating the intelligent sleeping device 102 communicatively coupled to a mobile device 120 to encourage the child 130 to follow a nighttime routine in real-time, according to one embodiment.

As shown in FIG. 6A, the mobile device 120 may be communicatively coupled to the intelligent sleeping device 102. The parent of the child 130 may set a bedtime of 7:30 pm for the child 130. Before going to bed, the parent may have set a number of activities for the child to perform, such as getting into his nighttime pajamas, brushing his teeth and reading a short story and gradually going to sleep at 8 pm.

The parent may set the intelligent sleeping device 102 to play a favorite lullaby of the child while preparing to sleep.

At 7:30 pm, the robotic personality 104 of the intelligent sleeping device 102 may start to yawn and call out the child's name. The robotic personality 104 may prompt the child 130 to go to his bedroom and get into his pajamas as shown in circle ‘A’ of FIG. 6A. The intelligent sleeping device 102 may capture the child's activity and send a notification 504 to the database 118 of the sleep management server 112. The child's activity is saved in the activity log 502 of the particular child in the database 118. Further, the robotic personality 104 may prompt the child to go to brush his teeth as shown in circle ‘B’ of FIG. 6A. In between, the robotic personality 104 may display animated characters that may interact with the child and/or play a song.

FIG. 6B is a continuation of the implementation view 650B of FIG. 6A illustrating the next steps of the child to follow the nighttime routine, according to one embodiment.

Once the child 130 has finished brushing his teeth, the intelligent sleeping device 102 may encourage the child to get into his bed as shown in circle ‘C’ of FIG. 6B. Further, the intelligent sleeping device 102 may project a beautiful night light 610 for the child to create a sleeping environment 602. The beautiful night light 610 may make the child feel drowsy as shown in circle ‘D’ of FIG. 6B. The intelligent sleeping device 102 may display an animated character to start a real-time interaction 604 and play his favorite nighttime lullaby (e.g., music 606) in low voice as selected by the parent's recommendations as shown in circle ‘E’ of FIG. 6B and prompt the child to get into his bed. The soothing audio-visual projection may allow the child to smoothly drift into sleep without much effort.

FIG. 6C is a continuation of the implementation view 650C of FIG. 6B illustrating the further steps of the child to follow the nighttime routine, according to one embodiment.

The beautiful night light 610 and the music 606 may gradually put the child 130 to sleep. The intelligent sleeping device 102 may then automatically dim the light (e.g., dim light 608) shown in circle ‘F’ of FIG. 6C. Circle ‘G’ of FIG. 6C shows a night light 610 display by the intelligent sleeping device 102 in the room for a peaceful night sleep for the child.

At a preset time in the morning, the intelligent sleeping device 102 may project a wonderful morning environment 612 showing clouds and sunshine with chirpy sounds in the background to wake the child up as shown in circle ‘H’ of FIG. 6C.

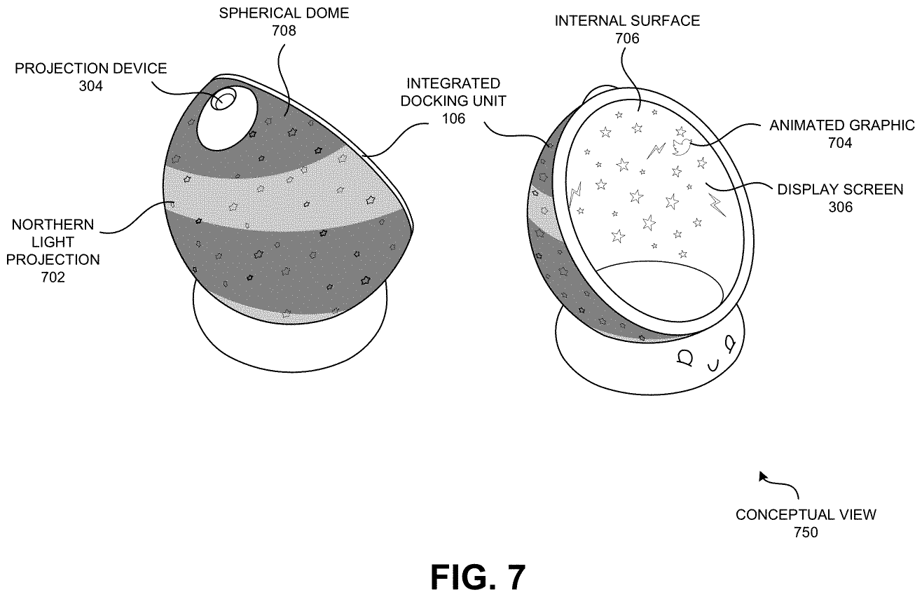

FIG. 7 is another conceptual view 750 of the sleep management system of FIG. 1 illustrating the northern light phenomena created by the intelligent sleeping device 102, according to one embodiment. The integrated docking unit 106 of the intelligent sleeping device 102 may project on the inside of the dome-like surface of the integrated docking unit 106. The internal surface 706 of the integrated docking unit 106 may act as a display screen 306. The projection device 304 at the base of the integrated docking unit 106 may project an animated graphic 704 at the internal surface 706 similar to a planetarium using real projection mapping.

In another embodiment, the external surface of the integrated docking unit 106 may act as a display screen 306. The projection device 304 at the base of the integrated docking unit 106 may project lights from the inside of the integrated docking unit 106 to the external surface of the spherical dome 708 to show a northern light projection 702 on the surface.

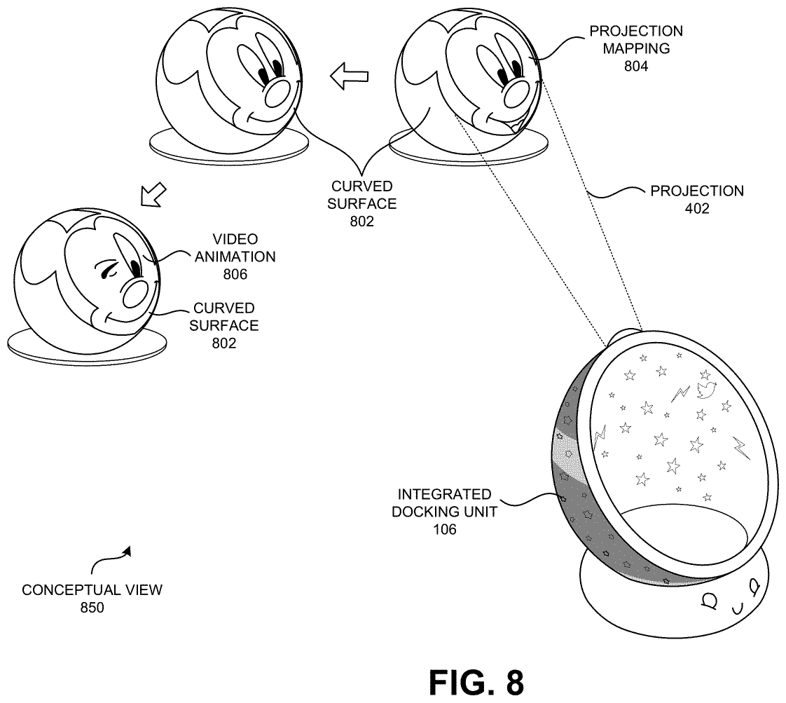

FIG. 8 is a conceptual view 850 of the sleep management system of FIG. 1 illustrating the rear projection mapping 804 on a curved surface 802 by the intelligent sleeping device 102, according to one embodiment.

As shown in FIG. 8, the integrated docking unit 106 may be programmed to turn an object, often circular and/or irregularly shaped small indoor objects (e.g., automated core unit 108, a globe, a semi-sphere, etc.), into a display surface for video projection. The integrated docking unit 106 may be programmed to display and/or project animation and/or a video film on any curved surface 802 using projection mapping 804. The rear projection mapping 804 may allow the integrated docking unit 106 to project accurately on a curved surface 802, such as a globular structure and/or a curved screen. “Any curved surface” may be implied that the face can be shaped to any character. The integrated docking unit 106 may be designed to project objects and/or graphic (e.g., animation) onto the curved surface 802 such that the object and/or the graphic wraps around the curved surface 802 and molds into their shape, turning common objects into interactive 3D displays. The rear projection mapping 804 may allow the video and/or animation 806 to be mapped onto the curved surface 802, turning common objects—such as globular structure (e.g., a toy, a globe, etc.) and/or a curved screen 802 into interactive displays. The curved surface 802 may become a canvas, with graphics being projected onto the surface, playing off of the surface's shape and textures to create a delightful experience of light and illusion for the child 130.

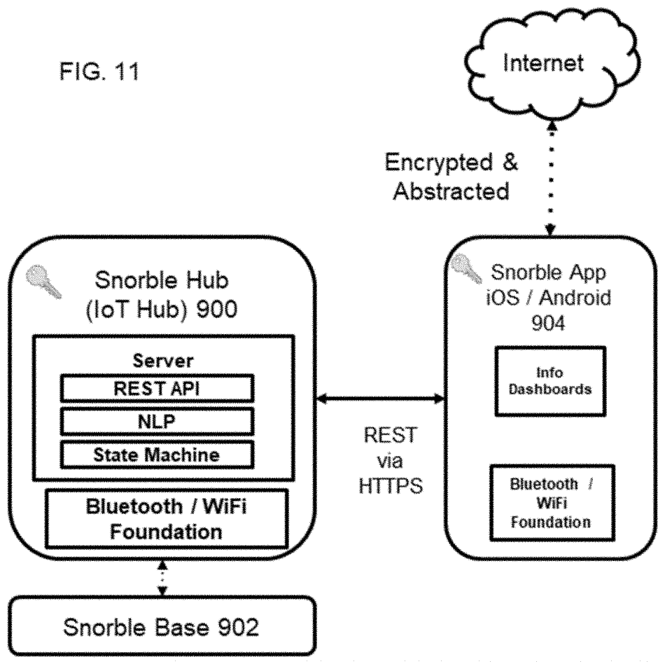

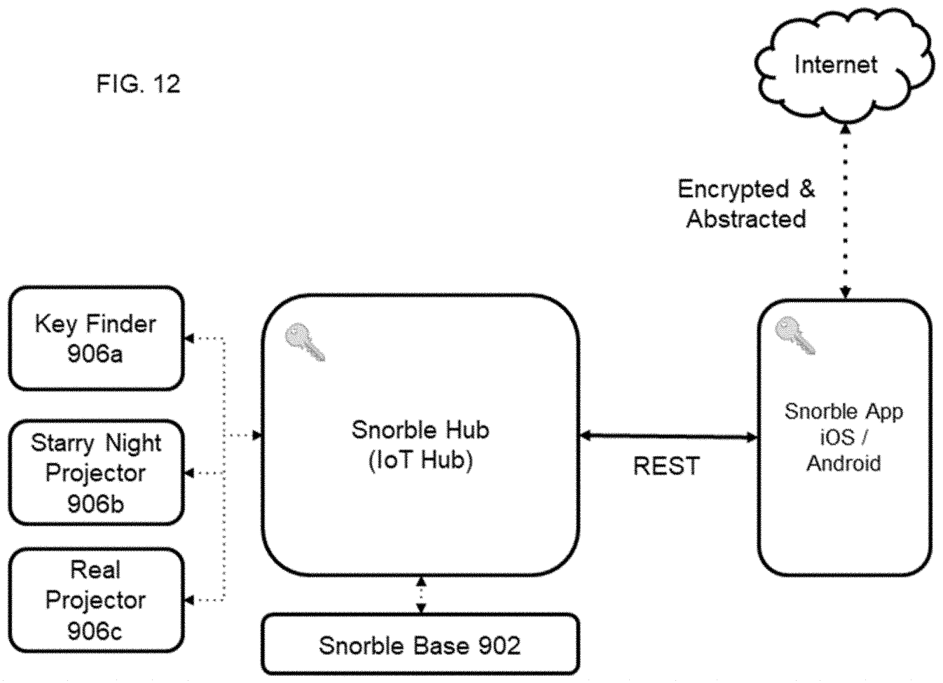

FIG. 11 and FIG. 12 provide diagrams of further representative embodiments of systems in accordance with the present disclosure.

With reference to FIG. 11, a hub 900 can be a standalone device that has no connection to the internet. In this implementation, hub 900 can obtain information through a connected app 904, by way of a smartphone, for example, that connects to the Internet and to the hub 900. The hub 900 can also serve as an IoT hub managing communication with add-on devices. A server of the system (not pictured) on a computer network, such as via the Internet, is responsible for orchestrating the functions of the hub 900. Such functions can include interfacing with a microphone (audio input) and passing the audio stream to a Natural Language Processing (NLP) software module that translates sound to text. The server is then responsible for passing the text and other inputs to a (e.g., Python State Machine) that determines an appropriate video to play. The server can then play (e.g., by streaming) the appropriate video by sending the video stream to the video output and audio stream to the audio output. The NLP module can be a proprietary Automated Speech Recognition (ASR) model developed by Applicant to recognize children's voices. The Python State Machine, in this example, takes a list of words and environmental inputs such as date, time and sensor (e.g haptic) and produces the correct video to play. The REST API can provide an interface for the Snorble App to interact with and can include the option to (i) update the configuration of the system, such as the core unit or base, (ii) update software on the system, (iii) update video content, (iv) retrieve activity history, (v) register additional devices, (vi) communicate with system devices, and the like. The app 904 is configured to connect to the Internet via the mobile device (iOS or Android, for example). The app also facilitates connection between a smartphone, for example, and the hub 900 by way of the REST API. The app can connect for the first time, during system setup for example, by way of a WiFi Access Point or Bluetooth. Once the connection is established a further method can be used to communicate, such as through a local or wide area network. Once the connection is made, commands can be issued directly through a REST API via HTTPS configured with a self-signed certificate, for example. Communication can be secured using a JSON Web Token or JWT. There can be a shared secret on the Snorble Hub and the Snorble App. This shared secret key can be used to create an accompanying hash (HMAC) to verify the authenticity of the message being received. The base 902 can be used as both a re-charging station and an ambient light projector. Base 902 can also be the first add-on IoT device in the system ecosystem.

With reference to FIG. 12, base 902 can be used as both a re-charging station and an ambient light projector. Additional IoT devices 906a-906c can be added to the local ecosystem such as a Key Finder, a Starry Night Projector and a Real Projector. Each IoT device can contain a communications module that supports both WiFi and Bluetooth for connectivity and discovery. Once connectivity has been established between the device and the IoT Hub, the device is then registered on the local WiFi network, for example. After the initial discovery and registration, devices and IoT Hub can communicate with each other through the Home WiFi. As each device is launched, a new version of the App is released to recognize that device.

In further accordance with the disclosure, a system can be provided wherein two core units 108 are in close proximity, such as when they occupy the same room and serve two different users (e.g., children). In this scenario, one of the core units 108 can manage the second core unit so as to prevent overlap and interference from one core device to the other. This can ensure that the correct core device responds to the unique owner of the device. When the core unit 108 does not sense another core unit 108 nearby, operation can return to normal.

In further accordance with the disclosure, a character kit can be provided. When the device's personality is “changed” via attachment of a new skin or outfit, additional functionality or capability can be unlocked and may be accessed via download from an approved e-commerce location. The system can instruct the connected peripherals to perform in a manner compatible with the new personality. This may include unlocking new functionality such as sounds and lights that are supportive of the new functionality, as well as how the peripheral acts when it is brought into close proximity to the core device 108, such as a certain lighting sequence or a buzzing sequence that serves as a greeting to the primary device. In the event that a device, e.g., 108 is brought into close proximity to another device, say for instance from a friend, the device can be caused to perform coordinated functions such as both units singing harmony or if there are 4 units they may sing like a barbershop quartet, for example.

Other peripherals can coordinate the core unit 108. This can provide, for example, supporting lights and music while the core unit is reading a story to a user. In another implementation, peripherals can provide back-up vocals to a song the core unit 108 is singing. Alternatively, a story could be told by a peripheral such as a charging base, with appropriate interactions at key times by the core unit 108. The timing of the output from the peripheral device can be controlled by the core unit 108. In some implementations, the algorithm for understanding specific sleep state can be achieved through a deployed machine learning model. The sensors that inform the algorithm can include beamforming microphone arrays as well as infrared motion sensing components that combine awareness of motion with validation via sound. Thermal imaging sensors may also be used. Ability to hear sounds at distance may be enhanced with one or more parabolic shaped surfaces with one or more microphones.

In some implementations, seamless sound scape routines can be provided to facilitate child sleep that include storytelling from the device, along with supporting soundscapes that give context to the story such as environmental sounds that would be compatible with the story. The system can be configured to restart the environmental soundscape when it detects that the child is imminently going to wake up and it is too early in the morning to wake up. Soundscapes can again fade away when sleep state is detected.

In some implementations, content can be selected based on a development level of the user. The device can be able to assess the developmental level of the user, such as a child, perhaps with the assistance of the caregiver. This may include evaluations of responses to provide content that is appropriate for that level of development.

In some implementations, one of the peripherals may be a bath toy, that communicates with the main device in a coordinated manner and is intended for use in the bath area. Metrics may be collected that are then passed over to the device 108 such as time in the bath, temperature of the water and the like. An additional peripheral may be a location device that may be added to a prized stuffed animal or other toy and will indicate its location to the main device to support a game of hide and seek, for example.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware circuitry (e.g., CMOS based logic circuitry), firmware, software or any combination of hardware, firmware, and software (e.g., embodied in a non-transitory machine-readable medium). For example, the various electrical structure and methods may be embodied using transistors, logic gates, and electrical circuits (e.g., application specific integrated (ASIC) circuitry and/or Digital Signal Processor (DSP) circuitry).

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the illustrative term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as illustrative. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as illustrative and for example only, with the following claims being entitled to their fullest breadth, including equivalents, under the applicable law.

What is claimed is:

1. A programmable interactive system to interact with a user to alter a user's behavioral patterns, comprising:
a processor;
at least one input sensor operably coupled to the processor to sense at least one sensor input;
at least one output device to output at least one stimulus to be observed by the user;
a core unit to interact with the user, the core unit being operably coupled to the processor; and
a non-transitory machine-readable medium embodying a set of machine readable instructions that, when executed by the processor, cause the system to:
detect one or more sensor inputs by way of the at least one sensor;
analyze said at least one or more sensor inputs to identify a parameter describing the status of the user; and
responsive to determining the parameter describing the status of the user, causing the at least one output device to change output of at least one stimulus that is observable by the user.

2. The system of claim 1, further comprising a docking unit configured to receive the core unit, wherein the docking unit and core unit are configured to communicate electronically with each other.

3. The system of claim 2, wherein the docking unit includes circuitry to project at least one visual output when the docking unit is coupled to the core unit onto a target surface.

4. The system of claim 3, wherein the non-transitory machine readable instructions further comprise instructions to output an audio output in synchronization with the visual output.

5. The system of claim 3, wherein the docking unit includes a projection device to project a visual image.

6. The system of claim 3, wherein the docking unit defines a parabolic surface and includes a microphone disposed in a location of the parabolic surface to focus incoming sound waves toward the microphone to enhance the system's ability to detect sounds made by the user.

7. The system of claim 1, wherein the core unit includes a reconfigurable exterior surface.

8. The system of claim 7, wherein the reconfigurable exterior surface includes an outer layer formed in the shape of a three dimensional object that can be removed from a frame of the core unit.

9. The system of claim 8, wherein the outer layer includes an identification tag that is detected by the core unit, wherein, responsive to detecting the identification tag, the processor selects and outputs at least one stimulus associated with the identification tag.

10. The system of claim 9, wherein the identification tag is an electronic identification tag including information stored thereon.

11. The system of claim 10, wherein the electronic identification tag includes a NFC chip or a RFID chip including digital information stored thereon.

12. The system of claim 9, wherein the identification tag is an optical identification tag including information encoded therein.

13. The system of claim 12, wherein the identification tag includes a QR code.

14. The system of claim 12, wherein the identification tag includes a bar code.

15. The system of claim 9, wherein the identification tag includes at least one visual indicium.

16. The system of claim 8, wherein the outer layer includes an identification tag that is detectable by a portable electronic device, wherein, responsive to detecting the identification tag, the processor selects and outputs at least one stimulus associated with the identification tag.

17. The system of claim 16, wherein the portable electronic device is a smart phone, and further wherein, responsive to detecting the identification tag, the smart phone accesses and downloads electronic files through a network connection and copies them to or installs them on the core unit.

18. The system of claim 8, wherein the system includes a plurality of different removable outer layers, wherein each said different removable outer layer is configured to be received by the core unit, and each said removable outer layer has a unique identification tag, wherein each said unique identification tag is identified by the system when the removable outer layer including said unique identification tag is mounted on the core unit, and further wherein, upon identifying said unique identification tag, a predetermined set of stimuli specific to said unique identification tag is selected that can be output by the system.

19. The system of claim 18, wherein each of the plurality of different removable outer layers has the appearance of a unique three dimensional figurine, and further wherein, responsive to identifying said unique identification tag, the system selects at least one file that includes information to cause the core unit to adopt behavioral characteristics associated with the unique three dimensional figurine.

20. The system of claim 19, wherein the unique three dimensional figurine corresponds to a unique action figure.

21. The system of claim 18, wherein the system provides additional functionality responsive to detecting mounting of a selected unique removable outer layer to the core unit.

22. The system of claim 1, wherein the system is configured to access updated configuration information from a remote server.

23. The system of claim 22, wherein the updated configuration information includes new visual information to project to the user.

24. The system of claim 22, wherein the updated configuration information includes new audio information to project to the user.

25. The system of claim 1, wherein the core unit is coupled to at least one processor, at least one memory, and at least one database.

26. The system of claim 25, wherein the at least one processor, at least one memory, and at least one database are onboard the core unit.

27. The system of claim 25, wherein the core unit includes at least one camera, at least one battery, at least one sensor, and at least one infrared detecting sensor.

28. The system of claim 25, wherein the core unit includes a visual projector therein and a projection forming a surface thereof, wherein the visual projector projects an image onto the projection screen responsive to user input.

29. The system of claim 28, wherein the projection screen is planar in shape.

30. The system of claim 28, wherein the projection screen is not planar in shape.

31. The system of claim 28, wherein the projection screen is spherical in shape.

32. The system of claim 28, wherein the projection screen is spheroidal in shape.

33. The system of claim 28, wherein the projection screen includes a section of compound curvature.

34. The system of claim 28, wherein the projection screen is formed by an intersection of curved surfaces.

35. The system of claim 25, wherein the core unit includes a haptic controller to process haptic input detected by sensors of the core unit.

36. The system of claim 1, wherein the machine readable instructions include instructions to recognize facial features or voice characteristics of the user.

37. The system of claim 1, wherein the machine readable instructions include instructions to interact with and respond to the user using natural language processing in real-time.

38. The system of claim 37, wherein the machine readable instructions include instructions to generate an audiovisual response in response to the status of the user.

39. The system of claim 37, wherein the machine readable instructions include a machine learning algorithm.

40. The system of claim 1, wherein the system is programmed to detect and analyze the user's voice to estimate the user's emotional state, and interact with the user by projecting a visual image responsive to the user's determined emotional state.

41. The system of claim 1, wherein the system is programmed to detect and analyze the user's voice to estimate the user's emotional state, and respond to the user by projecting an audio segment responsive to the user's determined emotional state.

42. The system of claim 1, further comprising a sleep management server that manages network resources by gathering data inputs related to sleep behavior of the user, analyzes the data inputs, and generates and sends at least one output to the user.

43. The system of 42, wherein the at least one output includes a recommendation to aid in sleep management decision-making for the user.

44. The system of 43, wherein the sleep management server includes machine readable instructions to maintain a real-time activity log to help develop and monitor a bedtime habit training of the user.

45. The system of 44, wherein the sleep management server includes instructions to provide a sleeping quality analysis of the user using a machine learning algorithm.

46. The system of claim 1, further comprising a plurality of peripheral devices configured to communicate wirelessly with the processor.

47. The system of claim 1, wherein the system is configured to detect using the at least one sensor when the user is restless or awakened.

48. The system of claim 47, wherein the at least one sensor includes at least one of a camera, a motion sensor, and a microphone.

49. The system of claim 47, wherein, responsive to determining if the user is restless or awakened, the system is configured to play soothing audio output.

50. The system of claim 1, wherein the system is configured to launch an interactive routine and interact with the user during the interactive routine.

51. The system of claim 50, wherein the routine is a bedtime routine and the system projects lighting conducive to sleeping during the interactive bedtime routine.

52. The system of claim 50, w wherein the routine is a bedtime routine and the system projects sounds conducive to sleeping during the interactive bedtime routine.

53. The system of claim 50, wherein the system alters the routine in response to detecting the state of the user.

54. The system of claim 50, wherein the system engages in a gamified routine to achieve a goal by the user.

55. The system of claim 54, wherein the goal is a household task, and the system provides instructions to the user to achieve the household task as the system detects the user taking actions in support of completing the task.

56. The system of claim 1, wherein the system is configured to launch an interactive wakeup routine and interact with the user during the interactive wakeup routine.

57. The system of claim 56, wherein the system projects lighting conducive to waking up during the interactive wakeup routine.

58. The system of claim 57, wherein the system projects sounds conducive to waking up during the interactive wakeup routine.

59. The system of claim 1, wherein the system is configured to emit synchronized sounds or light from at least one further peripheral device and the core unit when the at least one further peripheral device is within a predetermined proximity of the core unit.

60. The system of claim 59, wherein the at least one further peripheral device and the core unit provide complementary functions.

61. The system of claim 60, wherein the system engages in a gamified routine to facilitate interaction of a plurality of users, wherein each said user is associated with a respective core unit, and further wherein each said core unit includes a removable cover that resembles a unique three dimensional shape.

62. The system of claim 61, wherein the gamified routine includes a role playing routine.

63. The system of claim 1, wherein the machine readable instructions further include instructions to determine a specific sleep state of the user.

64. The system of claim 1, wherein the machine readable instructions further include instructions to read a narrative to the user while providing synchronized background sounds and lighting.

65. The system of claim 1, wherein the machine readable instructions further include instructions to play predetermined sounds during a bedtime routine, and to play said predetermined sound again if the system determines that the user is awakening during a predetermined time period.

66. The system of claim 1, wherein the machine readable instructions further include instructions to determine the developmental level of the user, and to provide audio and visual outputs responsive to the determined developmental level of the user.

67. The system of claim 1, wherein the machine readable instructions further include instructions to communicate with at least one peripheral device to obtain sensory inputs from the at least one peripheral device.

68. The system of claim 67, wherein the at least one peripheral includes a bath toy, and the system obtains bath water temperature input from the at least one peripheral device.

69. The system of claim 1, wherein the machine readable instructions further include instructions to communicate with at least one peripheral device to obtain location information from said at least one peripheral device.

70. A non-transitory machine-readable medium embodying a set of machine readable instructions that, when executed by a processor, cause the processor to carry forth any method described herein.

71. All methods as set forth herein.
